(19) United States
(12) Patent Application Publication
(10) Pub. No.: US 2026/0191829 A1
(43) Pub. Date: Jul. 9, 2026

(54) Compositions and Methods of Treating Neurological Conditions

(71) Applicant: DRI Biosciences Corporation, Frederick, MD (US)

(72) Inventor: Hao CHEN, Columbia, MD (US)

(73) Assignee: DRI Biosciences Corporation, Frederick, MD (US)

(21) Appl. No.: 19/128,416

(22) PCT Filed: Nov. 16, 2023
(86) PCT No.: PCT/US2023/080025

§ 371 Date: May 8, 2025

Related U.S. Application Data
(60) Provisional application No. 63/426,165, filed on Nov. 17, 2022.

(51) Int. Cl. A61K 31/4245; A61K 31/138; A61K 31/403; A61K 31/517; A61P 25/28; G01N 33/68; G01N 33/74; G01N 33/92

(52) U.S. Cl. CPC A61K 31/4245; A61K 31/138; A61K 31/403; A61K 31/517; A61P 25/28; G01N 33/6896; G01N 33/74; G01N 33/92; G01N 2800/2814; G01N 2800/2821

(57) ABSTRACT

Pharmaceutical compositions containing a dopamine Putamen reuptake inhibitor or a dopaminergic agonist, an adrenoceptor antagonist, and a pharmaceutically acceptable carrier, and methods for treating neurological diseases by administering the composition.

### CROSS REFERENCE TO RELATED APPLICATIONS

This application claims the benefit of U.S. Provisional Application 63/426,165, filed Nov. 17, 2022, which is incorporated herein by reference in its entirety.

### STATEMENT REGARDING FEDERALLY SPONSORED RESEARCH OR DEVELOPMENT

This invention was made with U.S. government support under award/contract numbers 1R43NS095422-01 and 1R43NS103696-01A1 awarded by the National Institutes of Health. The government has certain rights in the invention.

### TECHNICAL FIELD

The present invention relates to compositions and methods for treating neurological and neurodevelopmental disorders.

### BACKGROUND

Effective medication(s) for treating and improving neurological disease conditions are unmet medical needs. A number of therapeutic approaches have been developed to treat neurological and neurodevelopmental disorders, but these provided limited effectiveness. Thus, there continues to exist a problem of widespread suffering under these disorders with little hope of effective and lasting treatment. This unmet medical need is addressed by the instant invention, exemplary compositions and methods of which are described in more detail below.

### SUMMARY

In an aspect, the present invention reflects the discovery that these neurological disorders involve an excess in adrenergic activities and a deficit in dopaminergic activities.

In one aspect, the disclosure relates to a pharmaceutical composition comprising a dopamine reuptake inhibitor, an adrenoceptor antagonist, and a pharmaceutically acceptable carrier. In another aspect, the disclosure relates to a pharmaceutical composition comprising a dopaminergic agonist, an adrenoceptor antagonist, and a pharmaceutically acceptable carrier.

In one aspect, the disclosure relates to methods for treating the neurological and neurodevelopmental disorders by administering these pharmaceutical compositions to a subject in need thereof. Thus, one embodiment of the invention provides a pharmaceutical composition of a dopamine reuptake inhibitor or dopaminergic agonist plus an adrenergic receptor (“adrenoceptor”) antagonist for treatment of a neurological disease or condition.

In another aspect, the method (i.e., treatment use of the composition) also includes a step of measuring a biomarker level in the subject in need of treatment. This measuring step may occur before, during, and/or after treatment. In yet another aspect, this disclosure relates to a pharmaceutical kit containing a dopamine reuptake inhibitor or a dopaminergic agonist, an adrenoceptor antagonist, and reagents or instruments for assessing and measuring at least one or more biomarkers.

The biomarker used in the method or kit may be glucose metabolism and metabolite profiles, and/or lipid metabolism and related metabolite profiles, and/or hormone profile, and/or cytokine profile, or and/or cardiac function. The glucose metabolism biomarker may be, e.g., glucose, lactate, pyruvate, ratio of lactate to glucose, or rate of glucose disposal. The lipid metabolism and related metabolite profiles may be comprised of beta-hydroxybutyrate, or acetoacetate, and/or high-density lipoproteins, cholesterol, triglyceride, or free fatty acid, or rate of the beta-hydroxybutyrate or acetoacetate disposal. The hormone profile biomarker may be, e.g., cortisol or leptin. The cytokine profiles biomarker may be, e.g., IL-10, 11-4, IL-17, IL-23, or their corresponding ratios. The cardiac functions biomarker may be, e.g., heart rate, or heart rate variability.

### BRIEF DESCRIPTION OF THE DRAWINGS

FIGS. 1A and 1B show comparisons of tyrosine hydroxylase expressions in different postmortem human brain sections (pons/putamen; FIG. 1C). Comparing with the brain sections from individuals without neurodegenerative conditions, the frontotemporal dementia (FTD) brain sections showed increased expression levels of tyrosine hydroxylase in the pons and decreased expression of tyrosine hydroxylase in the putamen.

FIGS. 2A-2D show comparisons of lipid and glucose metabolomic activities between healthy controls and frontotemporal dementia patients. These analyses indicated that conditions of FTD are associated with metabolic dysregulation, i.e., increased lipid catabolism (beta-hydroxybutyrate is a product of lipid beta-oxidation) and altered glucose metabolisms.

FIGS. 3A-3F show comparisons of behavioral and lipid metabolic patterns of drug-treated and untreated (vehicle) P301L mice with the behavioral and lipid metabolic patterns of the Swiss Webster (SW) mice (a background strain of the transgenic P301L mice and used as healthy controls).

FIGS. 4A-4D show comparisons of learning and memory behaviors of the different drug-treated and untreated (vehicle) Fmr1 KO mice with FVB mice (a background strain of the transgenic Fmr1 KO mice and used as healthy controls).

### DETAILED DESCRIPTION

Those skilled in the art will understand that this invention may be embodied in many different forms and should not be construed as limited to the embodiments set forth in this application. Rather, these embodiments are provided so that this disclosure will fully convey the invention to those skilled in the art. Many modifications and other embodiments of the invention will come to mind in one skilled in the art to which this invention pertains having the benefit of the teachings presented herein.

In particular, the neurological and neurodevelopmental disorders may be fragile x syndrome (FXS), neurofibromatosis I (NF1), tuberous sclerosis (TS), Down syndrome (DS), or autism spectrum disorders (ASD), amyotrophic lateral sclerosis (ALS), frontotemporal dementia (FTD), progressive supranuclear palsy (PSP), multiple system atrophy, Alzheimer's disease (AD), Parkinson's disease (PD), Huntington's disease and other related neurological diseases or conditions. The disorders may also include, but are not limited to, neuropsychiatric conditions such as major depressive disorders (MDD), post-traumatic stress disorders (PTSD), and chemical dependency/substance abuse such as cocaine use related disorder. Collectively, these debilitating neurological conditions negatively impact patients'quality of life while imposing burdens on the affected families and society as a whole.

The present invention is based on the surprising results of studies using postmortem human brains and/or of animal models of human neurological disease conditions. These results indicated that catecholamine disequilibrium (such as but not limited to an excess in adrenergic and deficit in dopaminergic activities) is a component of disease-causing mechanisms. The present invention is also based on the result of studies treating animal models of human conditions simultaneously with a combination of agents—one agent enhancing dopaminergic activity and another agent attenuating adrenergic activity—can normalize the catecholamine disequilibrium thus mitigating disease symptoms.

The present inventions are also based on the findings that the same cerebral catecholamine disequilibrium affects a number of physiological functions. The physiological functions may include metabolisms such as an altered rate of glucose disposal (including both increased or decreased rates of glucose disposal), altered glucose metabolic pathways (gluconeogenesis, glycolysis and/or oxidative phosphorylation), and dyslipidemia (including increased lipolysis or catabolism). The functions may include alterations in cardiovascular functions such as altered heart rate (both tachy-and brady-cardia) and/or heart rate variability (HRV). And the same neurochemical functions may include immunological status between pro-and anti-inflammatory activities (for instance plasma levels of IL-10 or the ratio of IL-10 to IL-17).

The present invention is also based on the results of studies treating animal models of human conditions simultaneously with a combination of agents (e.g., an agent with pharmacological activities of enhancing dopaminergic actions and an agent attenuating adrenergic actions) has normalized the physiological activities (e.g., lipid or glucose metabolisms). Thus, the given physiological activities, e.g., metabolic, cardiac, and/or immunologic status, can be used as objective biomarkers for the evaluation of cerebral catecholaminergic equilibrium and treatment effects.

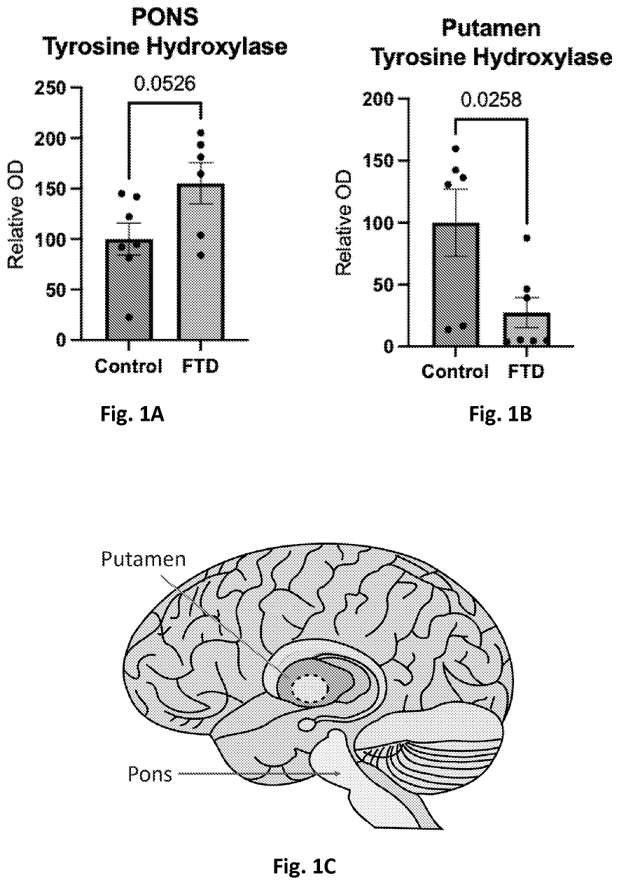

The overall profiles of tyrosine hydroxylase expressions in different brain regions of FTD patients indicates a pathophysiology of catecholamine disequilibrium. As shown in FIGS. 1A and 1B, the postmortem studies of neurodegenerative conditions represented by frontotemporal dementia (one of the conditions commonly called tauopathy) showed increased expression levels of tyrosine hydroxylase in the pons to which the locus coeruleus is a nucleus. Tyrosine hydroxylase is a rate limiting enzyme for catecholamine (dopamine, norepinephrine, and epinephrine) synthesis. The locus coeruleus is the primary cerebral region regulating the brain's adrenergic status. Increased tyrosine hydroxylase expression levels in the pons implies an elevated adrenergic activity in the brain. These same studies also showed a decreased expression of the same enzyme in the putamen, which is a section of the dorsal striatum and a primary cerebral region for regulating dopaminergic activity. Decreased tyrosine hydroxylase levels in this brain region indicates there is a loss of dopamine synthesis capacity and dopaminergic cells or loss of dopaminergic neurons in general, hence generating a deficiency in dopaminergic actions.

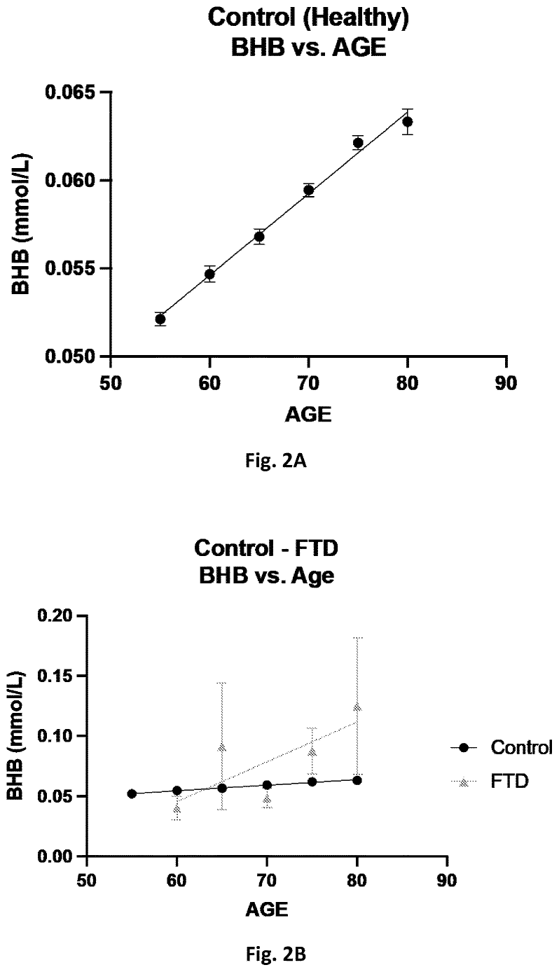

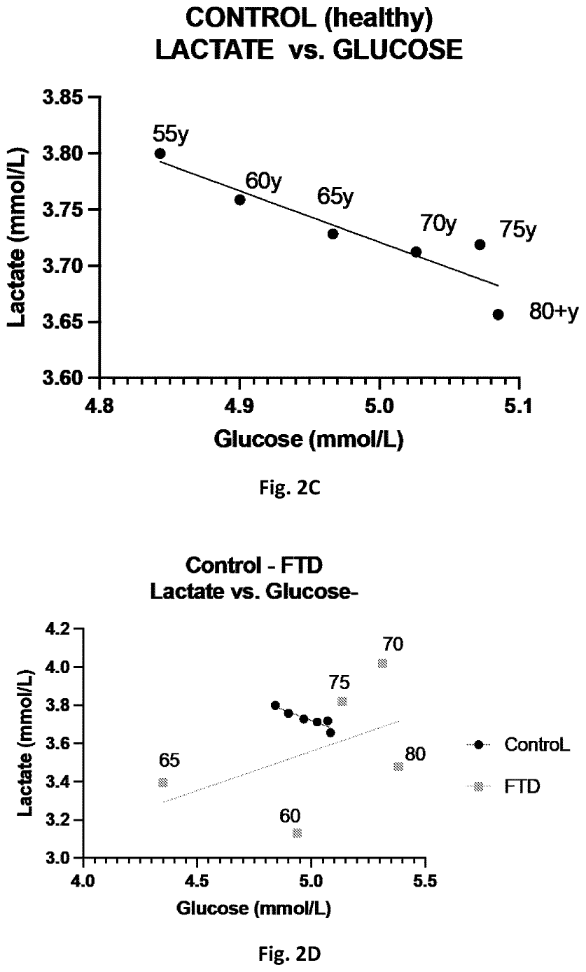

As shown in FIGS. 2A-2D, comparing with healthy individuals, the analysis of metabolomic data of frontotemporal dementia patients indicated metabolic abnormalities, e.g., increased lipid catabolism (beta-hydroxybutyrate is a product of lipid beta-oxidation) and dysregulated glucose metabolism. These changes in metabolism implicate an altered catecholamine equilibrium that is consistent with the differential expression of tyrosine hydroxylase noted above.

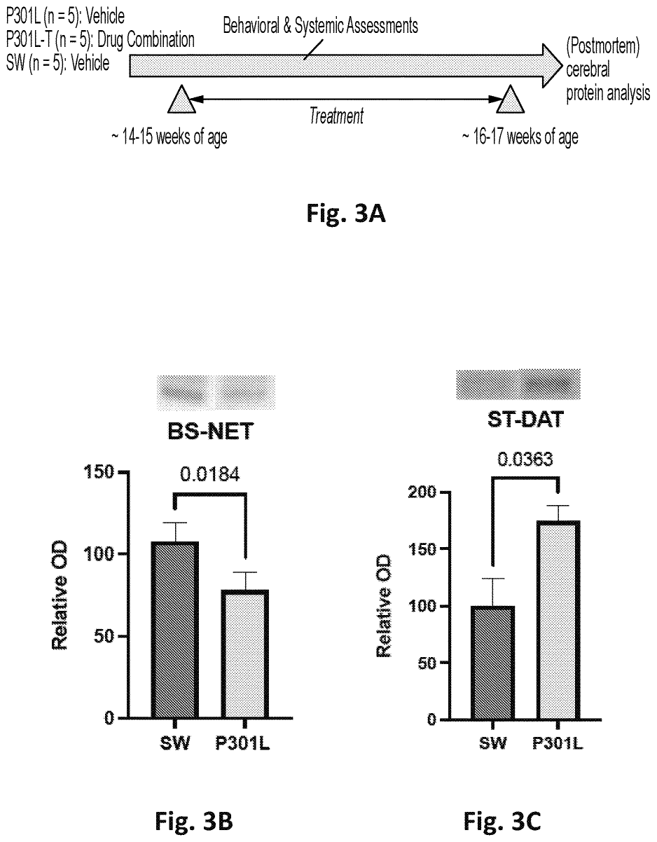

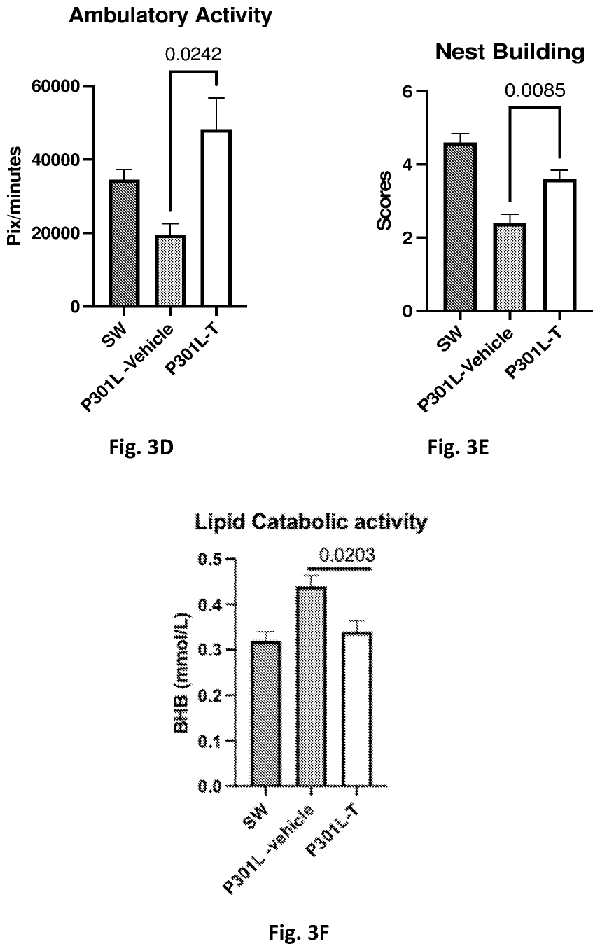

P301L mice, expressing the frontotemporal dementia-related mutation of the microtubule-associated protein tau (MAPT), are a murine model for human neurological diseases, such as frontotemporal dementia, Alzheimer's disease, or conditions collectively called tauopathy. As shown in FIGS. 3A-3F, the behavioral and lipid metabolic patterns of drug-treated and untreated P301L mice were compared with those of SW mice (SW=Swiss Webster mice, a background strain of for the transgenic P301L mice). Treating P301L mice with a combination of propranolol (an agent attenuating beta-adrenergic activity) and 3-(phenylpropyl)-sydnonimine-N-phenylcarbamoyl (an agent enhancing dopaminergic activity; see Chen et al., U.S. Pat. No. 8, 415,385) had normalized the behavior patterns and lipid metabolic activity of these P301L mice modeling human FTD conditions.

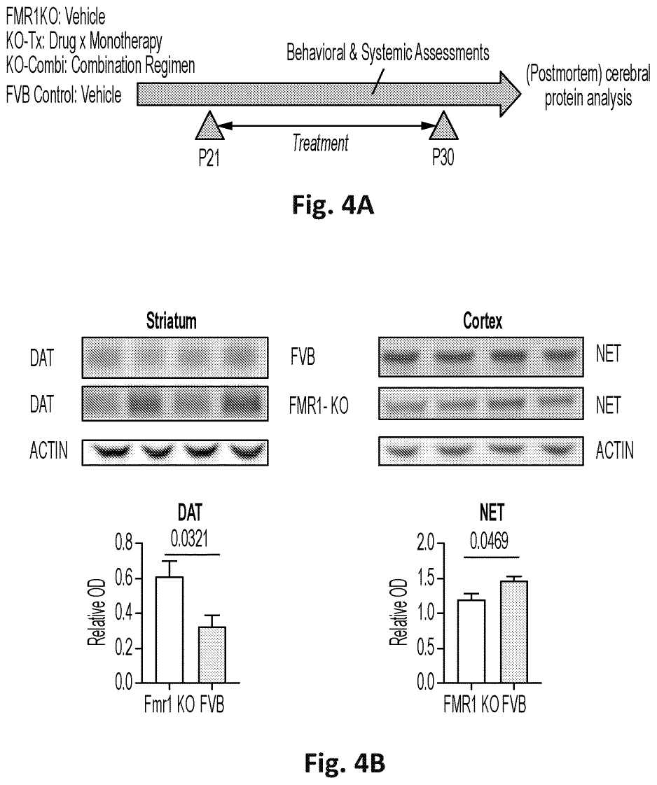

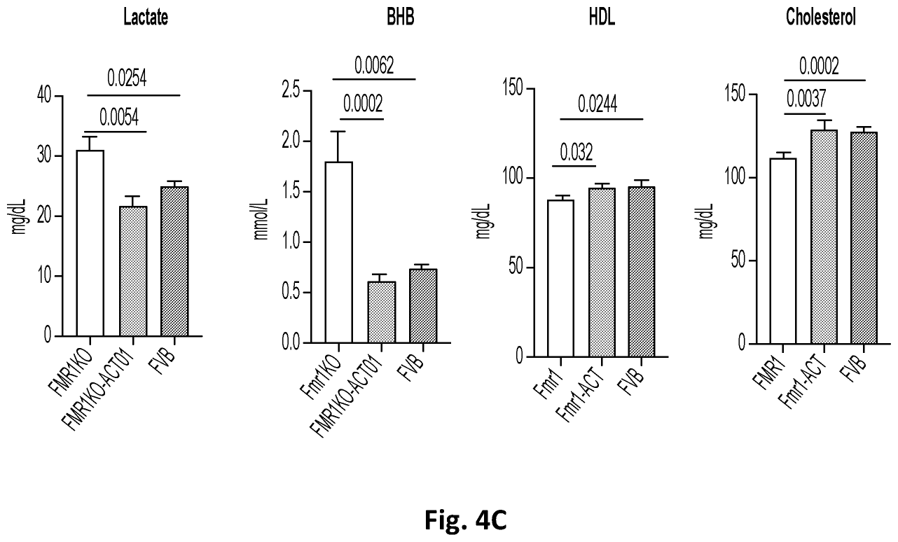

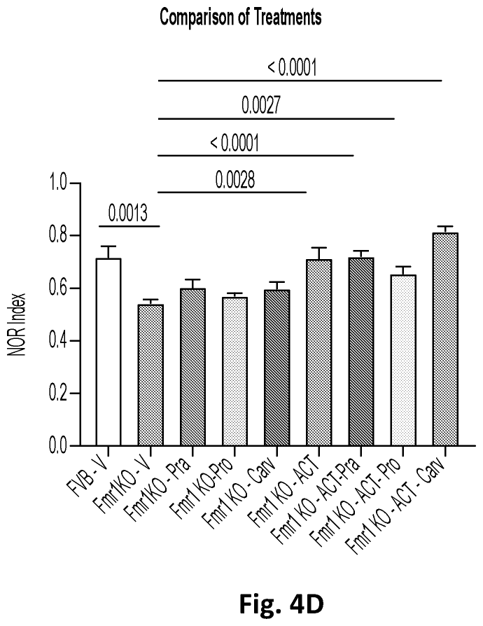

As shown in FIGS. 4A-4D, the study of Fmr1-KO murine brain, a rodent animal model of human fragile x syndrome and neurodevelopmental disorders, showed an increased expression of dopamine reuptake protein in the mouse striatum and reduced expression of norepinephrine reuptake protein at the murine brainstem (the locus coeruleus is a brainstem nucleus). The reuptake proteins regulate extracellular concentrations of different catecholamines. The differential expression patterns of these proteins indicated excessive adrenergic activity and a deficiency in dopaminergic actions, FIG. 4B.

The same knock-out animals also exhibit an increased glycolytic activity (increased plasma lactate), increase lipid catabolism (increased beta-hydroxybutyrate), and dyslipidemia (low HDL and cholesterol) resembling metabolic dysregulation in Fragile x humans. The altered metabolic activities indicated sympathetic dysregulation (i.e., catecholaminergic disequilibrium). Dopaminergic activity modulates sympathetic activation. Treatment of Fmr1 KO mice with ACT01, which specifically activates dopaminergic activities, normalized the glucose and lipid metabolisms, FIG. 4C.

The learning and memory behaviors of the drug-treated and untreated (vehicle) Fmr1 KO mice were compared with FVB mice (a background strain of the transgenic Fmr1 KO mice). The studies showed that the drug combinations, e.g., 3-(phenylpropyl)-sydnonimine-N-phenylcarbamoyl (aka ACT01 or ACT) (25 mg/kg) and propranolol (pro, 3 mg/kg), ACT01 (25 mg/kg), ACT01 (25 mg/kg) and prazosin (Praz, 2 mg/kg), and ACT01 (25 mg/kg) and carvedilol (Carv, 2 mg/kg), significantly (p<0.05) improved the Fmr1 KO mouse's learning and memory ability (NOR index=novel object recognition index). The drug combination effects are better than that of a single agent alone.

That is, treating Fmr1-KO mice with a combination of prazosin, carvedilol, or propranolol (agents attenuating adrenergic activity) with ACT01 (an agent enhancing dopaminergic activity; see Chen et al., U.S. Pat. No. 8, 415,385) rescued the animal model's learning ability and memory function and normalized the patterns of lipid and glucose metabolisms.

In a further embodiment, the dopamine reuptake inhibitor is chosen from but not limited to 3-(phenylpropyl)-sydnonimine-N-phenylcarbamoyl, 3-(p-methylbenzyl)-sydnonimine-N-phenylcarbamoyl, 3-(p-carboxybenzyl)-sydnonimine-N-phenylcarbamoyl, 3-phenethyl-sydnonimine-N-phenylcarbamoyl, 3-phenethyl-sydnonimine-N-(3′,4′-dichloro-phenyl)-carbamoyl, 3-(p-nitrophenethyl)-sydnonimine-N-(3′,4′-dinitro-phenyl)-carbamoyl, 3-(p-fluorobenzyl)-sydnonimine-N-phenylcarbamoyl, 3-benzyl-sydnonimine-N-phenylcarbamoyl, 3-phenethyl-sydnonimine-N-(p-chlorophenyl)-carbamoyl, 3-phenethyl-sydnonimine-N-(m-trifluoromethyl)-phenylcarbamoyl, 3-(3′,5′-difluorobenzyl)-sydnonimine-N-phenylcarbamoyl, 3-(m-fluorobenzyp-sydnonimine-N-phenylcarbamoyl, 3-(p-trifluoromethyl-benzyl)-sydnonimine-N-phenylcarbamoyl, 3-(p-tert-butylbenzyl)-sydnonimine-N-phenylcarbamoyl, 3-(p-methylbenzyl)-sydnonimine-N-(p′-trifluoromethyl-phenyl)carbamoyl, and 3-(p-methylbenzyl)-sydnonimine-N-(p-dimethylamino-phenyl)carbamoyl, mesocarb, altropane, amfonelic acid, amineptine, BTCP, 3C-PEP, DBL-583, Difluoropine, GBR-12783, GBR-12935, GBR-13069, GBR-13098, GYKI-52895, iometopane, modafinil, armodafinil, RTI-229, and vanoxerine.

One embodiment of the instant invention provides a pharmaceutical composition comprising a dopaminergic agonist and an adrenoceptor antagonist for treatment of a neurological disease or condition.

In a further embodiment, the dopaminergic agonist is chosen from but not limited to Apomorphine, Bromocriptine, Cabergoline, Ciladopa, Dihydrexidine, Dinapsoline, Doxanthrine, Epicriptine, L-dopa, Lisuride, Pergolide, Piribedil, Pramipexole, Propylnorapomorphine Quinagolide, Ropinirole, Rotigotine, Roxindole, Sumanirole.

In yet a further embodiment, the adrenoceptor antagonist is chosen from but not limited to propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, prazosin, terazosin, doxazosin, silodosin, alfuzosin, and tamsulosin.

In another embodiment, the dopamine reuptake inhibitor is chosen from 3-(phenylpropyl)-sydnonimine-N-phenylcarbamoyl (or mesocarb, or modafinil) and the adrenoceptor antagonist is propranolol, or carvedilol, or prazosin, or doxazosin, or tamsulosin.

One embodiment of the instant invention involves the method of adding adreno-blockers, such as beta adrenergic antagonists (e.g., propranolol) and or beta-alpha adrenergic antagonists (e.g., carvedilol or labetalol) in combination with agents that enhance dopaminergic activities, such as 3-(phenylpropyl)-sydnonimine-N-phenylcarbamoyl (Chen et al., U.S. Pat. No. 8, 415,385) or agent of similar pharmacological properties such as modafinil for control of a neurological disease state caused by catecholamine disequilibrium.

In a further embodiment of the instant invention, the dopamine activities are impacted by 3-(phenylpropyl)-sydnonimine-N-phenylcarbamoyl and the adrenergic activities are controlled by propranolol, or prazosin, or carvedilol in combination(s).

In a further embodiment of the instant invention,, the precise composition of the dopaminergic enhancing agent and adrenergic attenuating agent (and/or dosages) is determined hence formulated by the specific biomarker profiles of individual patient and/or his or hers stage of disease progressions.

The following examples serve to illustrate certain aspects of the disclosure and should not be construed as limiting the claims. The contents of all references, pending patent applications and published patents, cited throughout this application are hereby expressly incorporated by reference. In particular, the description and related drawings herein provided exemplary information to show the treatment effects of 3-(phenylpropyl)-sydnonimine-N-phenylcarbamoyl and propranolol combination animal behaviors but are not meant to be limiting for the scope of the present invention.

The methods described above may also include a step of measuring a biomarker level in the subject in need of treatment. It is also contemplated to have a pharmaceutical kit containing a dopamine reuptake inhibitor or a dopaminergic agonist, an adrenoceptor antagonist, and reagents or instruments for assessing and measuring at least one or more biomarkers.

The biomarker used in the method, and the biomarker contemplated for the kit, may be any numbers of combinations of glucose metabolism including metabolite profiles, lipid metabolism including metabolite profiles, hormone profile, cytokine profile, or cardiac function. The glucose metabolism biomarker may be, e.g., glucose, lactate, pyruvate, ratio of lactate to glucose, or rate of glucose disposal. The lipid metabolism and profiles biomarker may be, e.g., beta-hydroxybutyrate, acetoacetate, high density lipoprotein, or rate of the beta-hydroxybutyrate or acetoacetate disposal. The hormone profile biomarker may be, e.g., basal levels of insulin, cortisol (basal and evening cortisol levels) and or leptin (basal and postprandial leptin levels). The cytokine profiles biomarker may be, e.g., IL-10, Il-4, IL-17, IL-23, or their corresponding ratios. The cardiac functions biomarker may be, e.g., heart rate, or heart rate variability.

### EXAMPLES

### Example 1: Postmortem Study of Frontotemporal Dementia (FTD)

In comparison to individuals without neurodegenerations, the postmortem studies of neurodegenerative conditions represented by frontotemporal dementia (one of the conditions commonly called tauopathy) showed increased expression levels of tyrosine hydroxylase in the pons and decreased expression of the same enzyme in the putamen of individuals with FTD. The overall profile of tyrosine hydroxylase expressions in different brain regions indicated a pathophysiology of catecholamine disequilibrium.

The postmortem human brains, pons and putamen (a part of dorsal striatum) were stored at −80° C. and used for protein analyses. Tissue sections were homogenized (=100 μg tissues/μL of lysis buffer). Protein concentration was determined using the BCA assay (Pierce). The total protein concentration of the denatured lysates from each animal was adjusted to afford an identical total protein concentration (e.g., 1 μg/μL) so that identical amounts of the total protein were loaded onto each lane of a pre-cast gel (e.g., ≥5 ug/lane). Blots were developed with standard techniques and the images were captured and analyzed with ImageJ. The data were presented as relative optical density (“OD”) after normalization against the average expression levels of the healthy control.

As shown in FIG. 1A, there was a significant trend of increase in expressions of tyrosine hydroxylate at the pons of FTD patients in comparison to the controls (p=0.0526); in contrast, the expression levels of same enzyme were significantly reduced in the putamen of FTD patients (p=0.0258) (FIG. 1B). Locus coeruleus, a nucleus of the pons, governs of the cerebral status of norepinephrine; Putamen, a part of dorsal striatum, contributes to the central status of dopamine. The overall results of the protein analyses indicated an altered central catecholamine status, i.e., increased adrenergic activity and decreased dopaminergic activity.

### Example 2: Metabolomic Data of FTD Patients

The analysis of metabolomic data of frontotemporal dementia patients indicated metabolic abnormalities, e.g., increased lipid catabolism (beta-hydroxybutyrate is a product of lipid beta-oxidation) and increased glycolytic activities. The changes in metabolism implicate an altered catecholamine equilibrium, or excessive sympathetic activity through adrenergic activations and dopaminergic deficits.

Medical records including metabolomic data of FTD patients were extracted from the UK Biobank (UKBB) along with age-matched controls. Patients were separated into 6 age cohorts (<60, 60-64, 65-69, 70-74, 75-79, and 80+). Metabolomic data were used to establish the patterns of age-related lipid catabolic activities and to compare the same activity under the influence of FTD.

As shown in FIG. 2A, the analysis of plasma levels of beta hydroxybutyrate from the controls indicated a strong correlation between age and plasma beta hydroxybutyrate levels (R²=0.9924, p<0.0001, Y=0.0004622*X+0.02688). The analysis indicated that a “normal” metabolic aging involves a predictable course of increasing lipid catabolism Meanwhile, the same analysis using the FTD patient data also indicated a correlation between age (or disease progression) and levels of beta hydroxybutyrate within the FTD cohort (R²=0.5909, p<0.0094, Y=0.002753*X−0.1113).

In comparison to the controls, FIG. 2B, (Slopes: F=4.484, DFn=1, DFd=7, P=0.0720) 1), FTD presented an inflection of age or progression related acceleration on lipid catabolism which indicated an excessive sympathetic (adrenergic excess and dopaminergic deficit) activation under conditions of FTD.

As shown in FIG. 2C, the analysis of plasma levels of glucose and lactate from the controls indicated an age-dependent changes in plasma levels of lactic acid and glucose (R²=0.8453, p=0.0095, Y=−0.4567*X+6.004). The analysis indicated that a “normal” metabolic aging involves a predictable course of reduction in glycolytic activity.

Differing from the heathy subjects (controls), the same analysis using the data of FTD patients FIG. 2D, showed no correlation, but a trend suggesting age or disease progression related increase in glycolytic activity or dysregulation in glucose metabolism.

### Example 3: Studies of P301L (vs. SW) Mice and Examination of Treatment Effects

The experiment used P301L mice (Taconic Tau-Model 2508, JNPL 3(P301L) to model human conditions of FTD, tauopathy, Alzheimer's conditions (see Samaey, C., et al., Early Cognitive and Behavioral Deficits in Mouse Models for Tauopathy and Alzheimer's Disease. Front Aging Neurosci, 2019. 11: p. 335. Li, M. Z., et al., Intracellular accumulation of tau inhibits autophagosome formation by activating TIA1-amino acid-mTORC1 signaling. Mil Med Res, 2022. 9(1): p. 38. Wenger, K., et al., Common mouse models of tauopathy reflect early but not late human disease. Mol Neurodegener, 2023. 18(1): p. 10.). The experimental outline is provided in FIG. 3A. The mouse model expressed a transgene of the human P301L mutation of the microtubule-associated protein tau (MAPT) on a mixed background (Swiss Webster and C57/B6). During the 14^{th} to 15^{th} week (of age), the metabolite profiles and behavior patterns appeared to differ from the control animals. The timing of these observed differences is similar to the reported timing of behavioral changes reported previously (Samaey, et al, 2019).

First, (FIGS. 3B and 3C) in order to establish the model's relevancy to human conditions, i.e., deficiency in dopaminergic and excess in norepinephrine activities, the expression levels of norepinephrine transporter (NET) at mouse brainstem (BS-NET) and dopamine transporter (DAT) at striatum (ST-DAT) were analyzed, FIGS. 3A and 3B.

Mice of 15 weeks of age were sacrificed. Brains sections were isolated, frozen and stored until analysis. The expression levels of DAT at striatum and NET at brainstem were analyzed using the same methods described previously.

Intracellular over-expression of Tau causes mTORC1 activation (Li, M. Z., et al., Intracellular accumulation of tau inhibits autophagosome formation by activating TIA1-amino acid-mTORC1 signaling. Mil Med Res, 2022. 9(1): p. 38). Such activation causes an upregulation of dopamine transporter (DAT) and a downregulation of norepinephrine transporter (NET) expressions (Bermingham and Blakely, 2016). Increased expression of DAT reduces extracellular concentration of dopamine, and reduced expression of NET increases extracellular concentration of norepinephrine. As shown in FIGS. 3B and 3C, the P301L mice exhibited an upregulated expression of DAT at striatum and reduced expression at brainstem; thus created a conditions of adrenergic excess and dopaminergic deficit resembling human conditions of FTD.

The P301L mice were separated into 2 cohorts (n=5 each cohort), and one of them was treated with a drug combination of ACT01 (@25 mg/kg) and propranolol (@3 mg/kg), whereas the other P301L mice and age-matched SW mice were treated with vehicle as diseased and healthy controls respectively.

Treatment effects on animal behavior-Ambulatory activity (FIG. 3D): The mouse was placed in a previously habituated open field (box) and open field activity recorded with a fixed pixel resolution. The distance traveled (ambulatory activity) for a few minutes of the open field was assessed using a freeware application (Kinovea) as total pixel points crossed within a minute. Nesting behavior (FIG. 3E): Naive mice (individually housed) will be presented with a 2×2 cotton nestlet (VWR, 10279-140) at the beginning of the dark phase, the effect of treatment will be assessed by weighing the “un-processed” nestlet material.

As shown, FIGS. 3D and 3E, concurrent enhancement of dopaminergic activity by ACT01 and modulation of adrenergic activity through propranolol normalized the pattern of animal behaviors.

Treatment effects on animal lipid metabolisms—the analysis of the consequent metabolic change of the male P301L mice were also characterized. This study compared serum levels of beta-hydroxybutyrate of the drug treated, untreated P301L mice, with SW mice (SW=Swiss Webster mice, a background strain of for the transgenic P301L mice), FIG. 3. F. As shown, the concurrent mediation of different catecholaminergic activities with a dopamine reuptake inhibitor, ACT01, and a beta-blocker, propranolol, had normalized lipid catabolic activities.

### Example 4: Studies of Fmr1 Ko Mice (v.s FVB) and Examination of Treatment Effects

The study compared learning and memory behaviors of the different drug treated and untreated (vehicle) Fmr1 KO mice with FVB mice (a background strain of the transgenic Fmr1 KO mice; “FVB” refers to the susceptibility of FVB mice to Friend leukemia virus B). Male mice (FVB.129P2-Pde6b<+> Tyr<c-ch>Fmr1<tm1Cgr>/J (Stock No: 004624 |FMR1 KO)) were used as the FXS/autism model and age-matched male mice (FVB.129P2-Pde6b<+> Tyr<c-ch>/AntJ) were used as the controls (Bernardet, M. and W. E. Crusio, Fmr1 KO mice as a possible model of autistic features. Scientific WorldJournal, 2006. 6: p. 1164-76. Hays, S. A., K. M. Huber, and J. R. Gibson, Altered neocortical rhythmic activity states in Fmr1 KO mice are due to enhanced mGluR5 signaling and involve changes in excitatory circuitry. J Neurosci, 2011. 31(40): p. 14223-34. Willemsen, R. and R. F. Kooy, Mouse models of fragile X-related disorders. Dis Model Mech, 2023. 16(2).). The experimental outline and results are provided in FIGS. 4A-4D.

At about 5 years of age, fragile x patients exhibit macrocephaly such as enlarged putamen growth (Shen, M. D., et al., Subcortical Brain Development in Autism and Fragile X Syndrome: Evidence for Dynamic, Age- and Disorder-Specific Trajectories in Infancy. Am J Psychiatry, 2022. 179(8): p. 562-572. ; Williams, C. A., A. Dagli, and A. Battaglia, Genetic disorders associated with macrocephaly. Am J Med Genet A, 2008. 146A(15): p. 2023-37. ; and Hazlett, H. C., et al., Trajectories of early brain volume development in fragile X syndrome and autism. J Am Acad Child Adolesc Psychiatry, 2012. 51(9): p. 921-33). The mTOR is a key mechanism regulating growth. Excessive growth indicates excessive mTOR activity. The Tor-related mutations cause macrocephaly in ASD (Yeung, K. S., et al., Identification of mutations in the PI3K-AKT-mTOR signalling pathway in patients with macrocephaly and developmental delay and/or autism. Mol Autism, 2017. 8: p. 66.).

Activation of TOR activity was also reported in the rodent model, Fmr1 KO (Huang, W. C., Y. Chen, and D. T. Page, Hyperconnectivity of prefrontal cortex to amygdala projections in a mouse model of macrocephaly/autism syndrome. Nat Commun, 2016. 7: p. 13421. Sharma, A., et al., Dysregulation of mTOR signaling in fragile X syndrome. J Neurosci, 2010. 30(2): p. 694-702.). Albeit the relationships between the TOR activity and the expression levels of different catecholamine transporters (or reuptake proteins) is well-documented (Bermingham, D. P. and R. D. Blakely, Kinase-dependent Regulation of Monoamine Neurotransmitter Transporters. Pharmacol Rev, 2016. 68(4): p. 888-953.), in order to verify catecholaminergic dysregulations, the cortical expression of NET and striatal expression of DAT were analyzed, FIG. 4B. The protein analysis (western blot with method discussed above for FIGS. 1A and 1B) of the Fmr1 KO mouse (at postnatal P30) brain sections (isolated according to experimental procedures described previously) showed an increased expression of DAT and reduced expression of NET (different from the neurodegenerative human and animal brains, the protein expression levels are normalized against beta-actin). The upregulated DAT reduced the extracellular dopamine, whereas the downregulated NET increased the extracellular norepinephrine. The analysis established the fact that catecholamine status is similarly altered as in the previous discussion.

Treatment effects on rodent metabolic activities: As shown in FIG. 4C, these animals modeling Fragile X humans showed altered metabolic activities (e.g., lipid, etc.) similar to Fragile X humans (Berry-Kravis, E., et al., Cholesterol levels in fragile X syndrome. Am J Med Genet A, 2015. 167A(2): p. 379-84.), FTD patients, and P301L mice. Such alterations indicated an excessive sympathetic activation. Enhancement of dopaminergic action through ACT01 appeared to attenuate sympathetic activities and normalized the metabolic activities of the animals modeling Fragile x humans.

Unexpected drug-combination effects on rodent learning and memory: Novel object recognition test was used to assess treatment effects of different catecholaminergic mediations (including drug combinations) on cognitive behavior, i.e., learning and memory.

As shown by the experimental scheme (FIG. 4A), mice were weaned at P21 (21 days after birth), then immediately started on daily treatments with dopamine enhancing reuptake inhibitor, 3-(phenylpropyl)-sydnonimine-N-phenylcarbamoyl (or ACT01 or ACT), or adrenergic blockers, propranolol (Pro), prazosin (Pra), carvedilol (Carv), or their corresponding combinations with ACT01. ACT01 was provided via oral gavage at 25 mg/kg; Propranolol 2 mg/kg; Prazosin≈0.8 mg/kg; and Carvedilol was at ≈2 mg/kg. Vehicle was orally administered to the control groups. After 2 days of treatment, animals were subjected to the experimental protocol with daily drug treatment 2 hours before training/testing Novel Object Recognition: On day 1 (habituation) each animal was introduced in the middle of a plastic opaque-white arena and was allowed to freely explore for 20 min. On day 2 (sample phase), two identical objects were placed in two opposite corners and the mouse was introduced in the center of the arena for a 5-min training phase. Twenty-four hours later, the mouse was returned to the arena for a 5-min test phase, where one of the objects was replaced with a novel one. A preference index, a ratio of the amount of time spent exploring the novel object over the total time spent exploring both objects, was used to measure object recognition.

The studies showed, FIG. 4D, that the drug combinations, e.g., 3-(phenylpropyl)-sydnonimine-N-phenylcarbamoyl (ACT01 or ACT) by itself had positively enhance the animals (Fmr1-KO mice) cognitive; whereas the adrenergic blockers, by itself or individually may have suggestive trend but insignificant effects. The drug combination effects were significantly better than that of any single agent alone (p<0.05 is considered to be statistically significant).

What is claimed is:

1. A pharmaceutical composition comprising:
a. a dopamine reuptake inhibitor;
b. an adrenoceptor antagonist; and
c. a pharmaceutically acceptable carrier.

2. A pharmaceutical composition according to claim 1, wherein the dopamine reuptake inhibitor is 3-(phenylpropyl)-sydnonimine-N-phenylcarbamoyl, 3-(p-methylbenzyl)-sydnonimine-N-phenylcarbamoyl, 3-(p-carboxybenzyl)-sydnonimine-N-phenylcarbamoyl, 3-phenethyl-sydnonimine-N-phenylcarbamoyl, 3-phenethyl-sydnonimine-N-(3′,4′-dichloro-phenyl)-carbamoyl, 3-(p-nitrophenethyl)-sydnonimine-N-(3′,4′-dinitro-phenyl)-carbamoyl, 3-(p-fluorobenzyl)-sydnonimine-N-phenylcarbamoyl, 3-benzyl-sydnonimine-N-phenylcarbamoyl, 3-phenethyl-sydnonimine-N-(p-chlorophenyl)-carbamoyl, 3-phenethyl-sydnonimine-N-(m-trifluoromethyl)-phenylcarbamoyl, 3-(3′,5′-difluorobenzyl)-sydnonimine-N-phenylcarbamoyl, 3-(m-fluorobenzyl-sydnonimine-N-phenylcarbamoyl, 3-(p-trifluoromethyl-benzyl)-sydnonimine-N-phenylcarbamoyl, 3-(p-tert-butylbenzyl)-sydnonimine-N-phenylcarbamoyl, 3-(p-methylbenzyl)-sydnonimine-N-(p'-trifluoromethyl-phenyl)carbamoyl, and 3-(p-methylbenzyl)-sydnonimine-N-(p-dimethylamino-phenyl)carbamoyl, mesocarb, altropane, amfonelic acid, amineptine, BTCP, 3C-PEP, DBL-583, Difluoropine, GBR-12783, GBR-12935, GBR-13069, GBR-13098, GYKI-52895, iometopane, modafinil, armodafinil, RTI-229, or vanoxerine.

3. A pharmaceutical composition according to claim 2, wherein the dopamine reuptake inhibitor is 3-(phenylpropyl)-sydnonimine-N-phenylcarbamoyl or modafinil or mesocarb.

4. A pharmaceutical composition comprising:
a. a dopaminergic agonist;
b. an adrenoceptor antagonist; and
c. a pharmaceutically acceptable carrier.

5. A pharmaceutical composition according to claim 4, wherein the dopaminergic agonist is apomorphine, cabergoline, dihydrexidine, dinapsoline, doxanthrine, epicriptine (beta-dihydroergocryptine), L-dopa (levodopa and L-3,4-dihydroxyphenylalanine), lisuride, piribedil, pramipexole, quinagolide, ropinirole, rotigotine, roxindole, or sumanirole.

6. A pharmaceutical composition according to claim 5, wherein the dopamine agonist is L-dopa, or apomorphine, or rotigotine.

7. A pharmaceutical composition according to claim 1, wherein the adrenoceptor antagonist is propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, prazosin, terazosin, doxazosin, silodosin, alfuzosin, or tamsulosin.

8. A pharmaceutical composition according to claim 7, wherein the adrenoceptor antagonist is propranolol, carvedilol, prazosin, doxazosin, tamsulosin.

9. A method for treating a neurological disease, comprising administering a composition according to claim 1 to a subject in need thereof.

10. A method according to claim 9, wherein the neurological disease is fragile x syndrome (FXS), neurofibromatosis I (NF1), tuberous sclerosis (TS), Down syndrome (DS), or autism spectrum disorders (ASD), amyotrophic lateral sclerosis (ALS), frontotemporal dementia (FTD), progressive supranuclear palsy (PSP), Alzheimer's disease (AD), Parkinson's disease (PD), major depressive disorder (MDD), post-traumatic stress disorder (PTSD), or cocaine use related disorder.

11. A method according to claim 10, wherein the neurological disease is fragile x syndrome, autism spectrum disorders (ASD), amyotrophic lateral sclerosis (ALS), frontotemporal dementia (FTD), or Alzheimer's disease (AD).

12. A method according to claim 9, further comprising measuring a biomarker level in the subject, wherein the biomarker is:
a. glucose metabolism and metabolite profiles (e.g., glucose, pyruvate, lactate), or
b. lipid metabolism and profiles, or
c. hormone profile, or
d. cytokine profile, or
e. cardiac function.

13. A method according to claim 12, wherein the measuring step comprises measuring biomarker level of glucose metabolism and profiles thereof, and/or lipid metabolism and profiles thereof, and/or hormone profile (including dynamic profiles), and/or cytokine profiles or ratios thereof), or cardiac functions.

14. A method according to claim 12, wherein the glucose metabolism biomarker is glucose, lactate, or pyruvate, or ratio of lactate to glucose, or rate of glucose disposal.

15. A method according to claim 12, wherein the lipid metabolism and profiles biomarker is beta-hydroxybutyrate, or acetoacetate, and/or high density lipoprotein, triglyceride, or ratio of triglyceride to beta-hydroxybutyrate, or free-fatty acid to beta-hydroxybutyrate ratio, or rate of the beta-hydroxybutyrate or acetoacetate disposal.

16. A method according to claim 12, wherein the metabolite profiles consist of beta-hydroxybutyrate, lactate, and glucose.

17. A method according to claim 12, wherein the hormone profile biomarker is cortisol (basal and evening), leptin (basal and postprandial), or a cytokine profile.

18. A method according to claim 12, wherein the hormone profile biomarker is basal and/or postprandial leptin,

19. A method according to claim 16, wherein the hormone profile biomarker is cytokine profile biomarker IL-10, Il-4, and/or IL-17.

20. A method according to claim 12, wherein the cardiac function biomarker is heart rate or heart rate variability.
